(19) United States
(12) Plant Patent Application Publication
(10) Pub. No.: US 2011/0271413 P1
(43) Pub. Date: Nov. 3, 2011

(54) Stevia plant named 'T60'

(50) ;

(75) Inventor: Grania V. Garnighian, Prior Lake, MN (US)

(73) Assignee: Sweet Green Fields, LLC, Bellingham, WA (US)

(21) Appl. No.: 12/799,668

(22) Filed: Apr. 28, 2010

(51) Int. Cl. A01H 5/00

(52) U.S. Cl. PLT/226

(57) ABSTRACT

A new and distinct Stevia rebaudiana plant named ‘T60’ is described. The new variety is distinguished from other Stevia varieties by a high leaf concentration of rebaudioside A; its tall, bushy nature; its ovate leaves; and its long stem stature.

### BOTANICAL/COMMERCIAL CLASSIFICATION

Stevia rebaudiana L.

### VARIETY DENOMINATION

‘T60’.

### BACKGROUND OF THE INVENTION

Stevia rebaudiana is a shrubby perennial, growing up to 65-100 cm tall. It is a member of the Compositae family and originates from Paraguay. This species is grown commercially in Brazil, China, Paraguay, the United States, and other countries. The plants are grown so that sweet glycosides present in the leaves can be extracted for use as sweeteners. The sweetest of these glycosides is rebaudioside A (Reb A).

The development of new varieties of Stevia rebaudiana with high levels of Reb A is desirable. The new Stevia variety ‘T60’ described herein is such a variety.

The new ‘T60’ variety was asexually reproduced from callus through an in vitro tissue culture technique performed in Woodland, Calif.

### BRIEF SUMMARY OF THE INVENTION

The major characteristics exhibited by the new Stevia variety ‘T60’ that distinguish it from other varieties include a high concentration of total glycoside and high concentration of Reb A in the leaves from total glycoside. Other major characteristics that also distinguish the new Stevia variety ‘T60’ from other varieties include a tall, bushy stature that is resistant to high winds; spatulate-oblanceolate to ovate leaves; high yield foliage production. For example, ‘T60’ produced over 3000 lbs/acre in one harvest. Still other major characteristics that distinguish the new Stevia variety ‘T60’ from other varieties include being virus free, resistant to Fusarium ssp. and Sclerotinia ssp., drought resistant, and resistant to compact argilous soils. Another major characteristic exhibited by the new Stevia variety ‘T60’ that distinguishes it from other varieties is an average biological cycle of 90-115 days between 20-27 N,S Latt, giving the option of secondary harvest per annual cycle.

### BRIEF DESCRIPTION OF THE DRAWINGS

FIG. 1 depicts a color photograph of a fully mature ‘T60’ stevia plant near the end of the growing season.

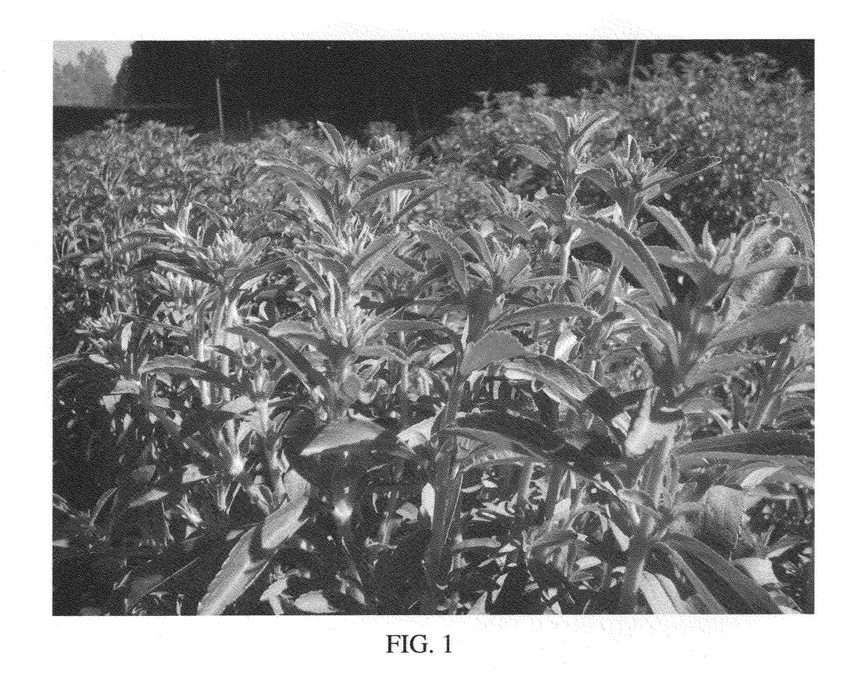

### DETAILED DESCRIPTION OF THE INVENTION

Four wild varieties of Stevia were imported from the Paraguayan government through the Ministerio de Agricultura and Ganaderia. These varieties were derived from a wild germ plasm in Paraguay, locally identified as ‘Criola’ material, and released by the Paraguayan government upon request for research and breeding. The four imported ‘Criola’ lines were named ‘IAN-135’, ‘IAN-142’, ‘IAN-228’, and ‘IAN-259’.

Table 1 depicts the glycoside and Reb A percentages for the ‘Criola’ lines described above.

TABLE 1

| | | | Plant | Length of | | | | |
|---|---|---|---|---|---|---|---|---|
| | | Flowering | Height | Width of | Leaves | Total | Total | Reb A to |
| Variety | Cycle | Days | (cm) | Leaves (cm) | (cm) | Glycoside | Reb A | Glycoside |
| IAN-228 | Early | 110 | 65 | 4 | 2 | 18.47 | 13.25 | 71.7% |
| IAN-135 | Medium | 180 | 82 | 4 | 2 | 16.56 | 8.06 | 48.7% |
| IAN-259 | Medium | 180 | 55 | 5 | 2 | 16.55 | 11.24 | 67.9% |
| IAN-142 | Late | 200 | 110 | 6 | 3 | 19.06 | 13.15 | 69% |

As a first step in the production of new Stevia varieties, the four ‘Criola’ lines were planted in vitro in tissue culture medium. The tissue culture material was multiplied by successively transferring plantlets in hormone-supplemented culture. Plantlets of each variety were planted into 2 inch plugs and then into 4 inch pots between December of year 1 and January of year 2. The plantlets were planted in a field in Woodland, Calif. between March and April of year 2. The plantlets were then allowed to flower, which occurred between July and August of year 2.

In August of year 2, anthers from the flowered plantlet were collected before anthesis. Approximately 200 anthers were collected from plantlets of each of the four ‘Criola’ lines. The anthers were then plated in vitro to derive haploid lines. The in vitro tissue culture medium induced callus formation from the plated anthers. Callus formation was observed in October of year 2. Haploid lines were created from the formed callus. The process of creating elite Stevia lines required that the chromosome count of each haploid line be doubled, since haploid lines cannot survive as plants. The full complement of chromosomes was regenerated by using successive colchicine treatments of 20 ppm, 40 ppm, and 60 ppm on both the callus and the shoots regenerated from the callus.

Every green callus that formed a diploid plantlet was transferred into a hormone-free medium for multiplication. Multiplication of the diploid plantlets resulted in the creation of a unique line of plants with unique characteristics distinguishable form the original four ‘Criola’ lines. These new Stevia lines were named T-lines. The T-lines were grown out in 2 inch plugs, then transferred to 4 inch pots, and then planted in the field in Woodland, Calif. The selected T-lines were shown to be superior to the parental stock. The selected T-lines either had higher total levels of glycoside, or higher levels of Reb A. Based on these characteristics, all callus material that gave rise to the new T-lines was collected. Genetically unique lines were developed from this callus material.

To test for percentage of Reb A production, the new T-lines were planted from tissue culture into 2 inch plugs and then transplanted into 4 inch pots. By the age of six weeks, this second generation of T-line plants had grown to about 10 inches in height in the pots. At six weeks the T-line plants were sampled to obtain HPLC data on the percentage of Reb A production. This was done even though the percentage of glycosides found in the leaf material of the new T-lines plants would not be as high as expected levels in mature plants. The levels of Reb A in these new T-line Stevia plants were as high as 89.2% of total glycoside levels. After the HPLC data was obtained, the T-line Stevia plants were planted in the field in Woodland, Calif., and grown to maturity. At maturity, the production rates of glycosides were evaluated.

All new T-lines were grown out in greenhouses in Woodland, Calif. for planting in the field, to allow for further testing and breeding for seed.

Of the new Stevia T-lines, three varieties were selected that produced high levels of Reb A. The production of high levels of Reb A in these Stevia T-lines will allow large amounts of Reb A to be harvested. The present Stevia variety ‘T60’ is one of the three selected T-lines developed by the methods described herein. The Stevia variety ‘T60’ was derived from the T-line ‘T6’. The ‘T6’ line was derived from ‘Criola’ line ‘IAN-135’.

Table 2 depicts HPLC data on the percentage of glycoside and Reb A for samples of the Stevia variety ‘T60’.

TABLE 2

| Samples of mature | Total | Total | Reb A to |
|---|---|---|---|
| ‘T 60’ Stevia plants | Glycoside | Reb A | Glycoside |
| 09-0144 | 15.18 | 12.58 | 82.8% |
| 09-0154 | 16.96 | 14.39 | 84.8% |
| 09-0165 | 14.58 | 12.00 | 82.3% |
| 09-0166 | 15.05 | 12.46 | 82.8% |

The HPLC data shows that the average percentage of Reb A to Glycoside in Stevia variety ‘T60’ was 83.2%. This is an increase of at least 11.5% over the ‘Criola’ lines. The ‘T60’ variety also showed an increase of 34.5% over ‘IAN-135’, from which ‘T60’ is derived.

### DETAILED BOTANICAL DESCRIPTION

The Stevia variety ‘T60’ is a perennial herb with filiform deep roots. The stems are erect, slender, and 65-100 cm tall. The stem easily produces secondary shoots (suckers) from its base, dies off and is renewed annually. The stem also produces numerous lateral branches, thus, forming a more-or-less roundish and dense crown.

The crushed leaves exude a strong odor, and all the green parts of the plant taste sweet. The leaves are simple, opposite, sub sessile, internodes, 2-4 cm long. Blades are subcoriaceous, very variable in shape and size, ovate, 2-3 cm long, 0.6-1 cm wide, apex obtuse, base cuneate, and margins entire serate on the upper half. Three primary veins arise from the leaf base raised and prominent on the blade's lower side, immersed on the upper side, and with secondary venation reticulate.

The blades in dry state are olive-green to brownish green in color (RHS 147A-147B) and usually darker on the upper side. Both surfaces are subscabrous with black glandular dots on the lower side, the leaves sub sessile or the petiole to 3-4 mm long. The capitula are arranged into loose, paniculate corymbose inflorescences at the terminal ends of the branches, peduncle 1-4 cm long, very slender, pedicels of each capitulum are slender and 1-4 mm long, bracts are linear-lanceolate, and 1-2 mm long. Each capitulum is enveloped by an involucre, light green on the lower half (RHS 142A), and yellowish (RHS 142C) on the upper half. The stems at maturity are brown-reddish (RHS 35A-35B)

The Stevia variety ‘T60’ has 5 phyllaries that are finely hairy, green (RHS 129B) when fresh, linear to subulate, 4-5 mm long, and acute to rounded at apex. Each capitulum is made up to 5 disk florets greenish white (RHS 155C). The seeds are achenes bearing numerous, equally long papus awns.

The Stevia variety ‘T60’ expresses high concentrations of total glycosides, with a total of rebaudioside A concentration of greater than 80%. The Stevia variety ‘T60’ has an average biological cycle of 90-115 days.

We claim:

1. A novel and distinct variety of Stevia plant named ‘T60’ having the characteristics described and illustrated herein.
